(19) United States
(12) Patent Application Publication
(10) Pub. No.: US 2017/0049970 A1
(43) Pub. Date: Feb. 23, 2017

(54) NEEDLE SAFETY SHIELD

(71) Applicant: New Injection Systems Ltd, Ipswich, Suffolk (GB)

(72) Inventors: Stephen Dunne, Ipswich, Suffolk (GB); Douglas Arthur Emmott, Woodbridge, Suffolk (GB)

(21) Appl. No.: 15/307,894

(22) PCT Filed: May 1, 2015
(86) PCT No.: PCT/GB2015/051306

§ 371 Date: Oct. 31, 2016

(30) Foreign Application Priority Data
May 1, 2014 (GB) 1407682.2

(51) Int. Cl. A61M 5/32

(52) U.S. Cl. CPC A61M 5/3271; A61M 5/3202; A61M 2005/3247; A61M 2005/3267

(57) ABSTRACT

A needle safety shield assembly for an injector comprises: a needle shield (231) for shielding a needle of the injector; and a shield locking means comprising a track (233, 236) adapted to engage with a runner (224), the track comprising at least one unidirectional barrier (232, 237) through which the runner can pass; in which movement of the needle shield to expose the needle for use and to subsequently re-shield the needle causes movement of the runner (224) along the track (233, 236) through the at least one unidirectional barrier (232, 237) and into a locked position in which further movement of the runner and needle shield is prevented.

The invention relates to a needle safety shield assembly for an injector, which may help to prevent needle-stick injuries to users. The invention also relates to a single-use injector comprising the needle safety shield assembly, a method of operating the needle safety shield assembly and a method of operating the single-use injector.

### BACKGROUND

Due to their sharp hypodermic needles, injectors pose a risk both to healthcare professionals as well as self-users. In particular, needle-stick injuries are a well known occupational hazard for healthcare workers due to the risk of transmitting blood-borne viruses such as hepatitis B and C and the human immunodeficiency virus (HIV).

In order to prevent needle-stick injuries to users, needle shields have been developed by the medical industry. Needle shields may be active or passive; active systems require activation of the safety mechanism by a clinician after injection, whereas passive systems automatically shield the needle after injection.

One type of passive needle shield includes a spring which pushes a shield forwards in order to cover the needle before and after use of the injector. As the shield is pushed against a patient's skin, the needle shield is pushed back relative to the needle until the needle extends beyond the shield and into the injection site. When the injector is removed from the injection site, the spring pushes the shield back so that it once again covers the needle.

### DESCRIPTION OF THE INVENTION

The invention provides a needle safety shield assembly for an injector, a single-use injector, a method of operating a needle safety shield assembly and a method of operating a single-use injector, as defined in the appended independent claims, to which reference should now be made. Preferred or advantageous features of the invention are set out in dependent sub-claims.

Thus, a needle safety shield assembly for an injector may comprise: a needle shield for shielding a needle of the injector; and a shield locking means comprising a track adapted to engage with a runner, the track comprising at least one unidirectional barrier through which the runner can pass. Movement of the needle shield to expose the needle for use and to subsequently re-shield the needle may cause movement or travel of the runner along the track through the at least one unidirectional barrier and into a locked position in which further movement of the runner and needle shield is prevented. The runner may be static in relation to the needle shield, in which case the track move to cause the runner to travel along the track. Alternatively, the runner may move with the needle shield to cause movement of the runner along the track. In the locked position it is preferable that the runner is located such that further movement of the runner is physically inhibited. It is preferable that the assembly is arranged such that movement of the shield can only occur with a consequent movement of the runner with respect to the track, and if the runner is prevented from moving with respect to the track then movement of the shield is prevented. Thus, by moving the runner into a locked position, the shield is locked in a position in which the needle is shielded. An injector comprising the needle shield assembly may, therefore, be a single use injector in which the needle can only be exposed for use on a single occasion before the shield assembly is locked in place.

The movement of the needle safety to a locked position in which further movement of the needle shield is prevented is advantageous because it prevents further exposure of the injector needle after use. This is beneficial because it reduces the risk of cross-contamination and/or needle-stick injuries. The locking of the needle shield also assists with the identification of used injectors and the safe destruction thereof.

The needle shield may be arranged to be movable between: a first shield position in which the needle is shielded and the shield is capable of being moved to expose the needle; a second shield position in which the needle is exposed for use and the shield is capable of being moved to re-shield the needle; and a third shield position in which the needle is re-shielded and the needle shield is prevented from further movement to expose the needle. The needle shield is preferably biased by a biasing means such as a spring towards a position in which the needle is shielded. Thus, the needle shield assembly may automatically shield the needle after use of the injector.

In one embodiment, the shield locking means comprises at least one track comprising at least one unidirectional barrier, the at least one track adapted to engage with the runner, movement of the needle shield being associated with movement of the runner such that in the first shield position the runner is engaged with the at least one track, on movement between the first shield position and the second shield position the runner passes along the at least one track, and on movement between the second shield position and the third shield position the runner continues to pass along the at least one track and through the unidirectional barrier into a locked position in which further movement of the runner and needle shield is prevented. The at least one track may be a single track. The at least one track may be a track having a first portion and a second portion extending in different directions. The at least one track may be more than one track, for example two tracks, that extend in different directions. Where there is more than one track the runner should be able to pass between the tracks.

In one embodiment, the shield locking means comprises a first track and a second track having a unidirectional barrier, the first and second tracks adapted to engage with the runner, movement of the needle shield being associated with movement of the runner such that in the first shield position the runner is engaged with the first track, on movement between the first shield position and the second shield position the runner passes along the first track into the second track, and on movement between the second shield position and the third shield position the runner passes along the second track and through the unidirectional barrier into a locked position in which further movement of the runner and needle shield is prevented.

In one embodiment, the shield locking means comprises a first track having a first unidirectional barrier and a second track having a second unidirectional barrier, the first and second tracks adapted to engage with the runner, movement of the needle shield being associated with movement of the runner such that in the first shield position the runner is engaged with the first track, on movement between the first shield position and the second shield position the runner passes along the first track and through the first unidirectional barrier into the second track, and on movement between the second shield position and the third shield position the runner passes along the second track and through the second unidirectional barrier into a locked position in which further movement of the runner and needle shield is prevented.

The movement of the needle shield may be reciprocal along the needle axis. In other words, the needle shield may move backwards and forwards relative to the injector casing along the needle axis. The needle shield may be movable along the needle axis, but not along other axes. The needle shield may be tubular, or substantially tubular, in construction and may slide reciprocally in the direction of the needle axis to shield and expose the needle.

The term “unidirectional” is used to describe the way in which the barriers described herein allow the passage of the runner in one direction, but not in the opposite direction.

The unidirectional barrier may be a flexible arm which can deflect to allow passage of the runner in one direction, but which prevents passage of the runner in the opposite direction. The flexible arm may be a resilient arm or may be spring-loaded. Such a resilient or spring-loaded flexible arm may spring back to its original position once the runner has passed over the arm. The advantage of using flexible arms is that they can act as effective unidirectional barriers and they can be cheaply and easily manufactured, e.g. from plastic.

In needle safety shield assemblies comprising two unidirectional barriers, the first unidirectional barrier (in the first track) may be a flexible arm and/or the second unidirectional barrier (in the second track) may be a flexible arm, wherein each flexible arm can deflect to allow passage of the runner in one direction, but prevents passage of the runner in the opposite direction. One or both of these flexible arms may be spring-loaded.

An advantage of such a flexible arm in the first track is that it allows passage of the runner along the first track only when the needle safety shield assembly moves from a first shield position to a second shield position. This prevents accidental locking of the needle safety shield assembly prior to injection. Another advantage is that it may prevent the runner from returning back along the first track after it has passed over the flexible arm, and it may therefore help the runner to move into the second track on movement between the first shield position and the second shield position.

In another embodiment, the unidirectional barrier may be a flexible wall or a deflectable which can deflect to allow passage of the runner in one direction, but which prevents passage of the runner in the opposite direction. The first unidirectional barrier may be a flexible wall and/or the second unidirectional barrier may be a flexible wall, wherein each flexible wall can deflect to allow passage of the runner in one direction, but which prevents passage of the runner in the opposite direction.

An advantage of having a flexible wall in the first track is that it allows passage of the runner along the first track only when the needle safety shield assembly moves from a first shield position to a second shield position. This prevents accidental locking of the needle safety shield assembly prior to injection. Another advantage is that it may prevent the runner from returning back along the first track after it has passed over the flexible wall, and it may therefore help the runner to move into the second track on movement between the first shield position and the second shield position.

An advantage of flexible wall unidirectional barriers are that they can be manufactured simply and easily, and it may be moulded from plastic as one part with the other components of the needle safety shield assembly. Thus, additional machining steps may be minimised or eliminated.

The needle safety shield assembly may be made of moulded plastic. Moulded plastic provides cost and reliability benefits over other materials.

The needle safety shield assembly may be moulded as one, two or three parts. It is advantageous to mould the needle safety shield assembly from as few parts as possible because this simplifies the manufacturing process leading to cost savings.

An injector may comprise a needle safety shield assembly as defined above. Preferably, the injector is a single-use injector.

An advantage of an injector comprising a needle safety shield assembly as defined above is that it is easy to use. In particular, the needle shield is movable from a first shield position in which the needle is shielded to a second shield position in which the needle is exposed for use, and the injector is movable from the second position to a third position in which the needle is re-shielded. Another advantage of a single-use injector is that it prevents re-exposure of the needle, which can cause needle-stick injuries and/or cross-contamination.

The needle shield may be moved from the first shield position to the second shield position by applying pressure to the needle shield, e.g. pushing the needle shield towards the injector casing. For example, the needle shield may be moved from a first shield position to a second shield position by pushing the needle shield against an injection site (i.e. the patent's skin). The needle shield may be moved from the second shield position to the third shield position by reducing or removing the pressure applied to the needle shield, e.g. by pulling the needle shield away from the injection site (i.e. the patent's skin).

In one embodiment, the injector comprises a biasing means which acts on the needle shield to ensure that the needle is shielded when the injector is not in use. The biasing means may be a spring, e.g. a helical spring.

In the first shield position, the biasing means may push against the needle shield so that it shields the needle. Moving the needle shield to the second shield position may comprise applying a force on the needle shield which is in the opposite direction to the force applied by the biasing means, and which is greater than the force applied by the biasing means. Applying this force may act to compress the biasing means. Moving the needle shield from the second shield position to the third shield position may comprise reducing the force applied to the needle shield, such that the force is less than the force applied by the biasing means. This reduction in the force applied to the needle shield may allow the biasing means to push the needle shield into a position in which the needle is shielded.

The single-use injector may be a manual needle insertion injector or an automatic needle insertion injector. The single-use injector may be an auto-injector. The auto-injector may be a hand-held auto-injector. The single-use injector may comprise a pharmaceutical glass or plastic cartridge, or a pre-filled syringe.

The needle may be a hypodermic needle for parenteral administration of a liquid medicament. The needle safety shield assembly may itself maintain the hypodermic needle in sterile conditions until use. Alternatively, the injector may further comprise a cap, which maintains the hypodermic needle in sterile conditions until use. In one embodiment, the needle cap does not contact the needle. The production steps for producing an injector, needle shield arrangement and/or cap may be carried out in a sterile environment.

The track or tracks may be defined in the needle shield and the runner may be defined on the injector body or on a second member. In addition, the track or tracks may be movable with respect to the needle; and the runner may be static with respect to the needle. Alternatively, runner may be defined in the needle shield and the track or tracks may be defined on the injector body or on a second member. In addition, the runner may be movable with respect to the needle; and the track or tracks may be static with respect to the needle.

In one embodiment, the runner may be attached to the injector casing. In this embodiment, the first and second tracks may be on the needle shield. Alternatively, the runner may be on the needle shield and the first and second tracks may be on the injector casing.

The runner may be a substantially cylindrical member, e.g. a rod or a pin.

The single-use injector may comprise an actuation means, wherein a liquid medicament is delivered from the single-use injector upon actuation of the actuation means. For example, the actuation means may comprise a button. In this embodiment, the injector may need to be pushed firmly against the injection site and then a button depressed in order to initiate the injection. Alternatively, the auto-injector may be push-actuated, i.e. pushing the auto-injector firmly against the injection site may initiate injection.

Actuation of the auto-injector may occur on the opening of a normally-closed valve. For example, a pressurised liquid medicament may be retained within the injector by a normally closed valve such as an aerosol valve or a pierceable septum. Opening of the valve leads to delivery of the medicament.

The actuation means may comprise a slideable shuttle comprising a piercing element for piercing a pierceable septum of a medicament container. In this embodiment, pushing the auto-injector firmly against the injection site may push the slideable shuttle towards the pierceable septum until the piercing element pierces the septum and releases the liquid medicament.

The auto-injector may comprise: a medicament container defining a substantially cylindrical chamber containing a liquid medicament, a proximal end of the chamber being closed by a piston slidably located within the cylindrical chamber, and a distal end of the chamber being closed by a container seal spanning an opening at a distal end of the medicament container; biasing means coupled to the piston and acting to bias the piston towards the container seal, thereby pressurising the liquid medicament; a hypodermic needle for parenteral administration of the liquid medicament; needle safety shield assembly as defined above; and means for establishing fluid communication between the chamber and the hypodermic needle such that the pressurised liquid medicament is automatically delivered through the hypodermic needle when communication has been established.

In one embodiment, the container seal is a pierceable septum spanning the opening at the distal end of the container and the means for establishing fluid communication is a valve comprising a valve housing defining a bore, the valve housing coupled to the distal end of the container such that the pierceable septum is located at a proximal end of the bore, and a shuttle slidably retained within the bore, the shuttle comprising a piercing element for piercing the pierceable septum when the shuttle is moved towards the proximal end of the bore to establish fluid communication between the chamber and the hypodermic needle. The piercing element and the hypodermic needle may be formed by opposite ends of a double-ended needle located by the shuttle.

The needle safety shield assembly may engage with the piercing element causing it to penetrate the pierceable septum. The needle safety shield assembly may engage with the piercing element when the needle shield is moved from a first shield position to a second shield position, wherein engagement with the piercing element causes the piercing element to penetrate the pierceable septum. A biasing force, preferably applied by a helical spring, may then force the liquid medicament out of the container and into the patient.

A method of operating a single-use injector as defined above may comprise the steps of: (1) moving the needle shield from the first shield position to the second shield position; (2) actuating the actuation means such that a liquid medicament is delivered from the single-use injector; and (3) upon completion of liquid medicament delivery, moving the needle shield from the second shield position to the third shield position.

A method of operating a needle safety shield assembly as defined above may comprise the steps of moving the needle shield to expose the needle for use; and moving the needle shield to re-shield the needle.

Also provided is a method of operating a needle safety shield assembly for an injector, said needle safety shield comprising: a needle shield for shielding a needle of the injector; and a shield locking means comprising a track adapted to engage with a runner, the track comprising at least one unidirectional barrier through which the runner can pass; said method comprising: (a) moving the needle shield to expose the needle for use; and (b) moving the needle shield to re-shield the needle, wherein steps (a) and (b) cause movement of the runner along the track through the at least one unidirectional barrier and into a locked position in which further movement of the runner and needle shield is prevented.

Also provided is a method of operating a needle safety shield assembly as defined above, comprising the steps of: (a) moving the needle shield from the first shield position in which the needle is shielded to the second shield position in which the needle is exposed for use, wherein on movement between the first shield position and the second shield position the runner passes along the first track and through the first unidirectional barrier into the second track; and (b) moving the needle shield from the second shield position to a third shield position in which the needle is re-shielded and the needle shield is prevented from further movement to expose the needle, wherein on movement between the second shield position and the third shield position the runner passes along the second track and through the second unidirectional barrier into a locked position in which further movement of the runner and needle shield is prevented.

Also provided is a method of operating the needle safety shield assembly as defined above, comprising the steps of: (a) moving the needle shield from the first shield position in which the needle is shielded to the second shield position in which the needle is exposed for use, wherein on movement between the first shield position and the second shield position the runner passes along the first track and through the first unidirectional barrier into the second track; and (b) moving the needle shield from the second shield position to a third shield position in which the needle is re-shielded and the needle shield is prevented from further movement to expose the needle; wherein on movement between the second shield position and the third shield position the runner passes along the second track and through the second unidirectional barrier into a locked position in which further movement of the runner and needle shield is prevented.

A safety shield or an injector according to embodiments of this invention may be used in conjunction with any drug whether a solution or a suspension or a mixture of these of any viscosity and density. Any of the drugs listed below, either on its own or a mixture thereof, may be injected using an auto-injector as disclosed herein:

17-alpha hydroxyprogesterone caproate, Corticotropin (ACTH), Laronidase, Factor VIII, Von Willebrand Factor Complex, Alefacept, Apomorphine Hydrochloride, Darbepoetin Alfa, Nelarabine, Bevacizumab, Interferon beta-1a, 11 mcg, Interferon beta-1a, 33 mcg, Factor IX complex, Interferon beta-1b, Ibandronate Sodium, Botulinum Toxin, Protein C Concentrate, Alglucerase, Imiglucerase, Injection, Secretin, Synthetic, Human, 1 Microgram, Glatiramer actate, Decitabine, Desmopressin acetate, Idursulfase, Etanercept, Epoetin alfa, Anadalufungin, Cetuximab, Ethanolamine Oleate, Hyaluronic acid derivatives, Agalsidase beta, Factor IX non-recombinant, Factor IX recombinant, Factor VIII (human), Factor VIII (porcine), Factor VIII recombinant, Feiba VH, Immune globulin (intravenous) (IVIG), Enfuvirtide, Immune globulin (intravenous) (IVIG), Somatropin, Hepatitis B Immune, Globulin (intravenous) (IVIG), Trastuzumab, von Willebrand factor complex, Adalimumab, Insulin for administration through DME (i.e., insulin pump), Hyaluronic acid derivatives, Mecasermin, Gefitinib, Levoleucovorin calcium, Ranibizumab Injection, Pegaptnib, Urofollitropin, Micafungin, Botulinum toxin type B, Aglucosidase alfa, Galsulfase, Somatropin, Factor VIIa, Atacept, Hyaluronic acid derivatives, Hyaluronan derivative, Immune globulin (intravenous) (IVIG), Hemin, Peginterferon alfa-2a, Peginterferon alfa-2b, Epoetin alfa, Somatrem, Efalizumab, Interferon beta-1a, subq, Zoledronic Acid, Infliximab, Treprostinil, Fluocinolone acetonide, intravitreal implant, Zidovudine, Eculizumab, Lanreotide, Histrelin implant, Palivizumab, Hyaluronic acid derivatives, Temozolomide, Antithrombin III (Human), Natalizumab, Panitumumab, Immune globulin (intravenous) (IVIG), Azacitidine, Verteporfin, Hyaluronidase, Bovine, Preservative Free, Naltrexone Depot, Teniposide, Omalizumab, 90Y-Ibritumomab tiuxetan, ADEPT, Aldesleukin, Alemtuzumab, Bevacizumab, Bortezomib, Cetuximab, Dasatinib, Erlotinib, Gefitinib, Gemtuzumab, lmatinib, Interferon alpha, Interleukin-2, Iodine 131 tositumomab, Lapatinib, Lenalidomide, Panitumumab, Rituximab, Sorafenib, Sunitinib, Thalidomide, Trastuzumab.

Embodiments of the invention may also be used to deliver biologics or small molecule drugs including a wide range of medicinal products such as vaccines, blood and blood components, allergenics, somatic cells, gene therapy, tissues, and recombinant therapeutic proteins, and substances that are (nearly) identical to the body's own key signalling proteins may also be injected using the invention. Examples are the blood-production stimulating protein erythropoietin, or the growth-stimulating hormone named (simply) “growth hormone” or biosynthetic human insulin and its analogues.

Embodiments of the invention may also be used to deliver monoclonal antibodies. These are similar to the antibodies that the human immune system uses to fight off bacteria and viruses, but they are “custom-designed” (using hybridoma technology or other methods) and can therefore be made specifically to counteract or block any given substance in the body, or to target any specific cell type.

Embodiments of the invention may also be used to deliver receptor constructs (fusion proteins), usually based on a naturally-occurring receptor linked to the immunoglobulin frame. In this case, the receptor provides the construct with detailed specificity, whereas the immunoglobulin-structure imparts stability and other useful features in terms of pharmacology.

Embodiments of the invention may also be used to deliver any of the following:

Alpha1-Adrenergic Antagonists, Analgesic Agents, Anesthetics, Angiotensin Antagonists, Inflammtory Agents, Antiarrhythmics, Anticholinergics, Anticoagulants, Anticonvulsants, Antidiarrheal Agents, Antineoplastics and Antimetabolites, Antineoplastics and Antimetabolites, Antiplasticity Agents, Beta-Adrenergic Antagonists, Bisphosphonates, Bronchodilators, Cardiac Inotropes, Cardiovascular Agents Central Acting Alpha2-stimulants, Contrast Agents, Converting Enzyme Inhibitors, Dermatologics, Diuretics, Drugs for Erectile Dysfunction, Drugs of Abuse, Endothelin Antegonists, Hormonal Agents and Cytokines, Hypoglycemic Agents

Hypouricemic Agents and Drugs Used For Gout, Immunosuppressants, Lipid Lowering Agents, Psychotherapeutic Agents, Renin Inhibitors, Serotonergic Antagonist Steroids, Sympathomimetics, Thyroid and Antithyroid Agents, Vasodilators, Vasopeptidase Inhibitor.

The auto-injector may also be used to deliver any drug with indications for Rheumatoid arthritis or Multiple sclerosis, Hemophilia A or B, Vasculitis, Beta-thalassemia, Anemia, blood coagulation disorders, Von Willebrand disease, Sickle cell anemia, Solid Tumours, Leukemia, all cancers including liver, bladder, renal, esophageal, overian, breast, prostate, pancreatic, colorectal or lung, malignant melanoma, multiple myeloma, crohn's disease, ulcerative colitis, uveitisfabry disease, pompe disease, viral infections, HIV, Hepatitis A, B, C, Marburg virus, Wolman disease, Muscular dystrophy, botulism, muscular diseases, ebola virus, gout, acne, psoriasis, COPD, asthma, Alzheimer's, ALS, migraine, synovitis, fibrosis or any other indication.

Embodiments of the invention may also be used to deliver any drug approved and listed by the FDA in the USA or any other national or international agency. Additionally any generic or biosimilar or biobetter drug on the market or in development.

Embodiments of the invention may also be used to deliver any one of the following: Lipitor, a cholesterol-lowering statin drug, Nexium, an antacid drug, Plavix, a blood thinner, Advair, Abilify, an antipsychotic drug, Seroquel, an antipsychotic drug, Singulair, an asthma drug; Crestor, a cholesterol-lowering statin drug, Actos, a diabetes drug or Epogen, an injectable anemia drug.

Embodiments of the invention may also be used to deliver any other drug not listed above capable of being injected and available at present or being developed by any pharmaceutical company or any other company anywhere in the world.

Embodiments of the invention may be used to inject humans or animals.

### SPECIFIC EMBODIMENTS OF THE INVENTION

The invention will now be described with reference to the figures in which:

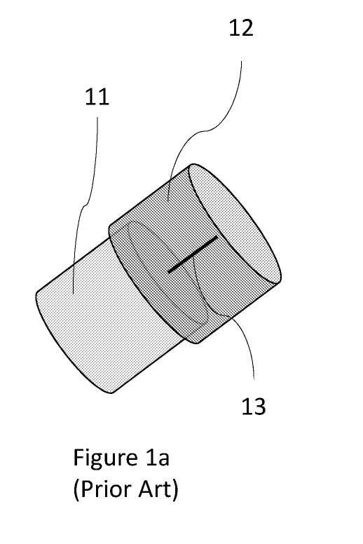

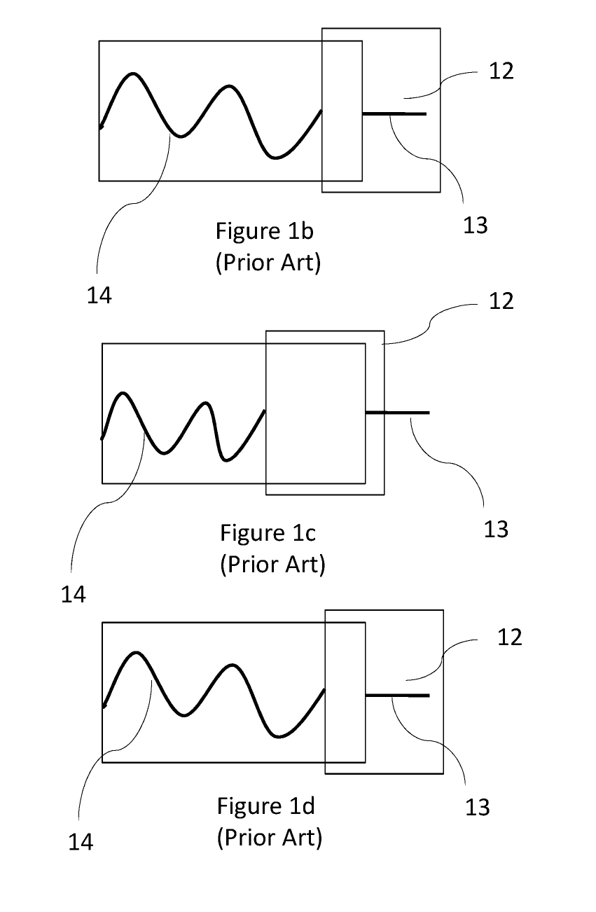

FIG. 1a is a schematic illustration of a typical needle shield concept as known in the prior art.

FIG. 1b is a schematic cross-sectional diagram illustrating the prior art needle shield of FIG. 1a with its needle shielded prior to use.

FIG. 1c is a schematic cross-sectional diagram illustrating the prior art needle shield of FIG. 1a with its needle exposed for use.

FIG. 1d is a schematic cross-sectional diagram illustrating the prior art needle shield of FIG. 1a with its needle shielded after use.

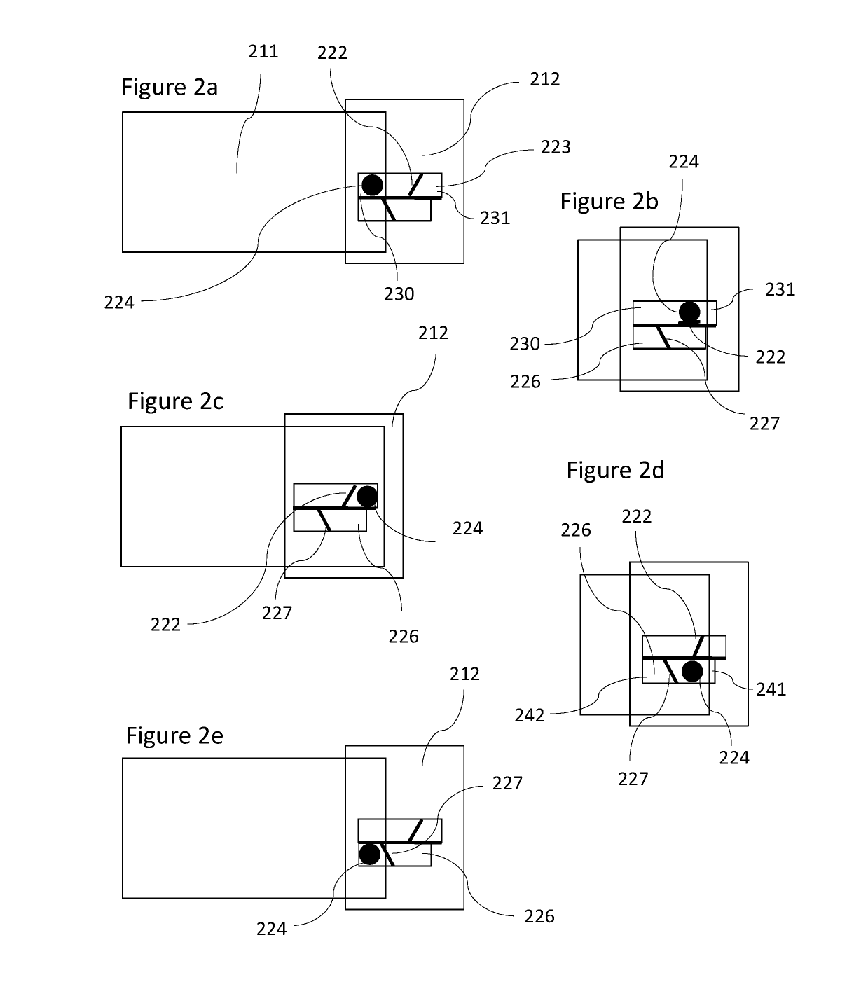

FIG. 2a shows a needle safety shield assembly according to an embodiment of the present invention.

FIGS. 2b to 2e illustrate the operation of the needle safety shield assembly of FIG. 2a.

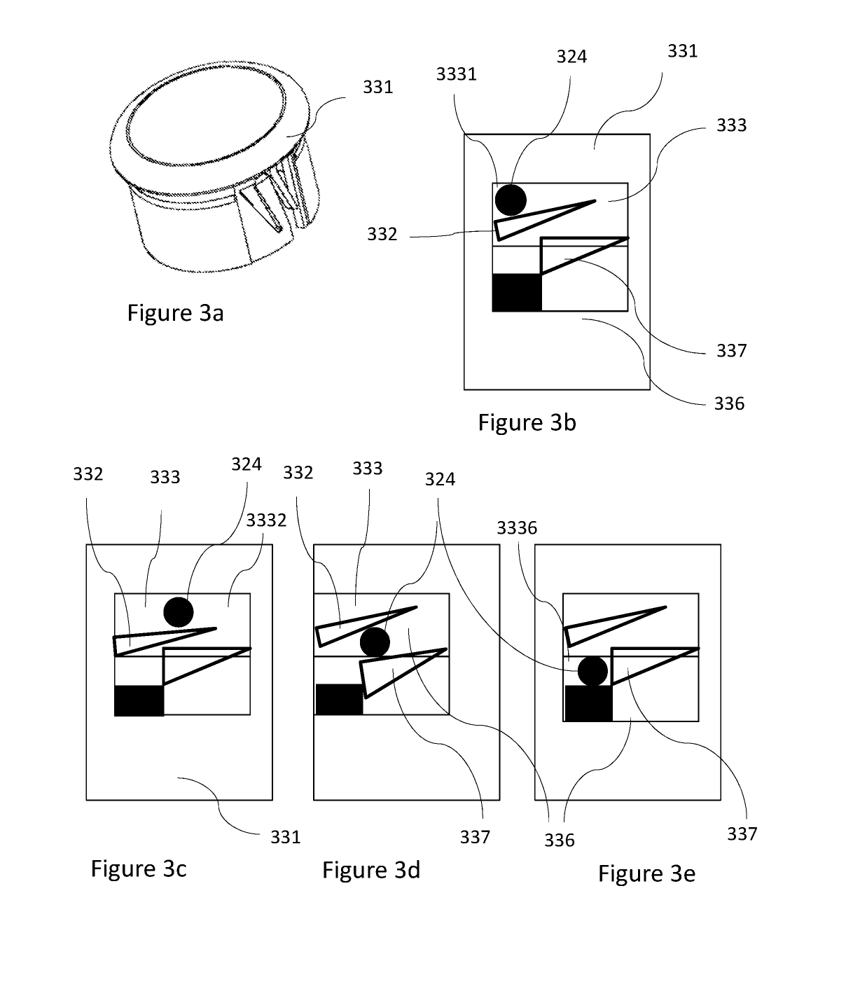

FIG. 3a shows a needle safety shield assembly according to the present invention.

FIGS. 3b to 3e illustrate the operation of the needle safety shield assembly of FIG. 3a.

FIGS. 1a to 1d show a typical prior art needle shield concept. In FIG. 1a, part of an injector casing 11 of an auto-injector has a needle 13 attached. The needle 13 is covered by a needle shield 12. As can be seen in FIG. 1b, a spring 14 pushes against the needle shield 12 in order to hold the needle shield 12 in its closed, or storage, position.

To use the prior art auto-injector, the needle shield 12 is pressed against a patient's skin. When the user pushes the needle shield against the injection site, the needle shield 12 is pushed back against the spring 14 (in the opposite direction as the spring's force) such that the needle 12 protrudes out of the needle safety shield assembly and penetrates the injection site (i.e. the skin), as shown in FIG. 1c.

As shown in FIG. 1d, when the injector needle is removed from the injection site (i.e. when the force applied to the injector during the injection procedure is reduced), the spring 14 pushes the shield 12 back to its original position to cover the needle.

FIGS. 2a to 2e illustrate a needle safety shield assembly according to an embodiment of the present invention. As can be seen in FIG. 2a, an injector casing or body 211 has a needle shield 212 attached. The injector comprises a needle (not shown) which is shielded by the needle shield 212. A spring is arranged to bias the needle shield towards a closed position in which the needle is shielded. The injector casing 211 defines, or is coupled to, an outwardly projecting runner 224, such as a lug, or pin. The needle shield 212 defines a first track and a second track with which the pin can engage. The first track 223 and the second track 226 lie adjacent to each other and are connected such that the pin can slide along the first track and into the second track. A first unidirectional barrier 222 is located in the first track 223, and a second unidirectional barrier 227 is located in the second track. The first track and the second track are substantially parallel.

When the safety shield is assembled, the pin 224 is located at a first end 230 of the first track 223. The first unidirectional barrier 222 is formed from a flexible spring loaded arm located in the first track 223.

As shown in FIG. 2b, when the shield 212 is pushed back to expose the needle for use, the pin 224 moves relative to the track such that the pin travels from the first end 230 of the first track 223 towards a second end 231 of the first track 223. As the pin travels from the first end of the first track to the second end of the first track it passes the first unidirectional barrier 222. The pin is thereafter prevented from returning to the first end 230 of the first track. When the injector has been used and the user removes the injector, the needle shield 212 starts to return to its closed position under the biasing action of a spring (not shown). The pin cannot travel back along the first track and is, instead, deflected into a first end 241 of the second track 260, as shown in FIG. 2d.

As shown in FIG. 2e, the return of the needle shield to re-shield the needle forces the pin 224 to travel along the second track 260 towards a second end 262 of the second track 260. As the pin travels along the second track, it passes the second unidirectional barrier 227, which in this embodiment is also in the form of a flexible spring loaded arm. The pin 24 becomes trapped behind flexible spring loaded arm 227, i.e. the pin cannot return along the second track towards the first end 261 of the second track. Thus, the pin is locked and cannot moved from its position at the second end 262 of the second track. The pin thereby prevents further movement of the shield by physically impinging the shield. Thus, in this position the shield 212 is in a locked position in which the injector needle is covered, thus protecting users from needle stick injuries. The injector cannot be re-used as the needle shield is locked in a closed position.

FIGS. 3a to 3e illustrate a needle safety shield assembly according a further embodiment of the present invention.

In FIG. 3a, a plastic moulded needle shield and track arrangement 331 is shown where the shield and spring loaded flexible tracks are moulded as one single integral part.

As can be seen schematically in FIG. 3b, the needle shield 331 has a first track 333 defined in part by a first flexible wall 332. A pin 324 is located at a first end 3331 of the first track 333. The pin 324 forms part of an injector casing that holds a needle (neither shown).

When the shield 331 is pushed back relative to the pin 324 to expose the needle for use, the first flexible wall 332 deflects to allow the pin 324 to travel along the first track 333. Once the pin has travelled beyond the flexible first wall 332 to a second end 3332 of the first track, the flexible wall returns to an undeflected position, thereby closing the first track and preventing the pin from returning along the first track.

After use, the shield 331 moves back again to re-shield the needle, forced by a spring (not shown). As the pin cannot travel back along the first track it is deflected into a second track 336. The second track 336 is defined in part by a second flexible wall 337. As the pin travels along the second track 336, the second flexible wall 337 deflects to allow the pin to travel to a second end 3336 of the second track 336. On passing the second flexible wall, the second flexible wall returns to an undeflected position, thereby locking the pin at the second end of the second track. The shield is now locked, as shown in FIG. 3e, and cannot be used again.

In both examples shown in FIG. 2 and FIG. 3 there may be some rotational movement of the shield relative to the injector casing.

Other embodiments are possible. Preferably the shield and tracks and arms are moulded as one component.

The needle safety shield assembly may be used with a manual needle insertion injector or an automatic needle insertion injector. The relative motion of the shield and needle holding part of the injector is the same in both instances.

1. A needle safety shield assembly for an injector, comprising:
a needle shield for shielding a needle of the injector; and
a shield locking means comprising a track adapted to engage with a runner, the track comprising at least one unidirectional barrier through which the runner can pass;
in which movement of the needle shield to expose the needle for use and to subsequently re-shield the needle causes movement of the runner along the track through the at least one unidirectional barrier and into a locked position in which further movement of the runner and needle shield is prevented.

2. A needle safety shield assembly according to claim 1 in which the needle shield is arranged to be movable between:
a first shield position in which the needle is shielded and the shield is capable of being moved to expose the needle;
a second shield position in which the needle is exposed for use and the shield is capable of being moved to re-shield the needle; and
a third shield position in which the needle is re-shielded and the needle shield is prevented from further movement to expose the needle;
characterised in that:
the shield locking means comprises at least one track comprising at least one unidirectional barrier, the at least one track adapted to engage with the runner, movement of the needle shield being associated with movement of the runner such that in the first shield position the runner is engaged with the at least one track, on movement between the first shield position and the second shield position the runner passes along the at least one track, and on movement between the second shield position and the third shield position the runner continues to pass along the at least one track and through the unidirectional barrier into a locked position in which further movement of the runner and needle shield is prevented.

3. A needle safety shield assembly according to claim 1 or claim 2 in which the needle shield is arranged to be movable between
a first shield position in which the needle is shielded and the shield is capable of being moved to expose the needle;
a second shield position in which the needle is exposed for use and the shield is capable of being moved to re-shield the needle; and
a third shield position in which the needle is re-shielded and the needle shield is prevented from further movement to expose the needle;
characterised in that:
the shield locking means comprises a first track and a second track having a unidirectional barrier, the first and second tracks adapted to engage with the runner, movement of the needle shield being associated with movement of the runner such that in the first shield position the runner is engaged with the first track, on movement between the first shield position and the second shield position the runner passes along the first track into the second track, and on movement between the second shield position and the third shield position the runner passes along the second track and through the unidirectional barrier into a locked position in which further movement of the runner and needle shield is prevented.

4. A needle safety shield assembly according to any of the preceding claims in which the needle shield is arranged to be movable between
a first shield position in which the needle is shielded and the shield is capable of being moved to expose the needle;
a second shield position in which the needle is exposed for use and the shield is capable of being moved to re-shield the needle; and
a third shield position in which the needle is re-shielded and the needle shield is prevented from further movement to expose the needle;
characterised in that:
the shield locking means comprises a first track having a first unidirectional barrier and a second track having a second unidirectional barrier, the first and second tracks adapted to engage with the runner, movement of the needle shield being associated with movement of the runner such that in the first shield position the runner is engaged with the first track, on movement between the first shield position and the second shield position the runner passes along the first track and through the first unidirectional barrier into the second track, and on movement between the second shield position and the third shield position the runner passes along the second track and through the second unidirectional barrier into a locked position in which further movement of the runner and needle shield is prevented.

5. A needle safety shield assembly according to any of the preceding claims in which movement of shield is reciprocal along the needle axis.

6. A needle safety shield assembly according to any of the preceding claims in which the unidirectional barrier is a flexible arm which can deflect to allow passage of the runner in one direction, but which prevents passage of the runner in the opposite direction.

7. A needle safety shield assembly according to claim 6 in which the flexible arm is spring-loaded.

8. A needle safety shield assembly according to claim 4 in which the first unidirectional barrier is a flexible arm and the second unidirectional barrier is also a flexible arm, wherein each flexible arm can deflect to allow passage of the runner in one direction, but prevents passage of the runner in the opposite direction.

9. A needle safety shield assembly according to claim 8 in which one or both of the flexible arms are spring-loaded.

10. A needle safety shield assembly according to any of claims 1-7 in which the unidirectional barrier is a flexible wall which can deflect to allow passage of the runner in one direction, but which prevents passage of the runner in the opposite direction.

11. A needle safety shield assembly according to claim 4 in which the first unidirectional barrier is a flexible wall and the second unidirectional barrier is also a flexible wall, wherein each flexible wall can deflect to allow passage of the runner in one direction, but prevents passage of the runner in the opposite direction.

12. A needle safety shield assembly according to any of the preceding claims in which the safety shield is made of moulded plastic.

13. A needle safety shield assembly according to any of the preceding claims, which is moulded as one, two or three parts.

14. A single-use injector comprising a needle and a needle safety shield assembly according to any one of the preceding claims.

15. A single-use injector according to claim 14 in which the track or tracks are defined in the needle shield and the runner is defined on the injector body or on a second member.

16. A single-use injector according to claim 15 in which the track or tracks are movable with respect to the needle; and the runner is static with respect to the needle.

17. A single-use injector according to any of claims 14-16 in which the runner is a pin which is attached to the injector body.

18. A single-use injector according to any of claims 14-17 in which the injector is a manual needle insertion injector or an automatic needle insertion injector.

19. A single-use injector according to any of claims 14-18 in which the single-use injector comprises actuation means, wherein a liquid medicament is delivered from the single-use injector upon actuation of the actuation means.

20. A single-use injector according to claim 19 in which the actuation means comprises a button.

21. A single-use injector according to any one of claims 14-20 comprising:
a medicament container defining a substantially cylindrical chamber containing a liquid medicament, a proximal end of the chamber being closed by a piston slidably located within the cylindrical chamber, and a distal end of the chamber being closed by a normally-closed valve, for example an aerosol valve or a container seal spanning an opening at a distal end of the medicament container;
biasing means coupled to the piston and acting to bias the piston towards the normally-closed valve, thereby pressurising the liquid medicament;
a hypodermic needle for parenteral administration of the liquid medicament;
a needle safety shield assembly according to any one of claims 1-13; and
means for establishing fluid communication between the chamber and the hypodermic needle such that the pressurised liquid medicament is automatically delivered through the hypodermic needle when communication has been established.

22. A single-use injector according to claim 21 in which the normally-closed valve is a container seal having a pierceable septum spanning the opening at the distal end of the container and the means for establishing fluid communication is a valve comprising a valve housing defining a bore, the valve housing coupled to the distal end of the container such that the pierceable septum is located at a proximal end of the bore, and a shuttle slidably retained within the bore, the shuttle comprising a piercing element for piercing the pierceable septum when the shuttle is moved towards the proximal end of the bore to establish fluid communication between the chamber and the hypodermic needle.

23. A single-use injector according to claim 22 in which the needle safety shield assembly engages with the piercing element when the needle shield is moved from the first shield position to the second shield position, wherein engagement with the piercing element causes the piercing element to penetrate the pierceable septum.

24. A method of operating a single-use injector according to any one of 19-23 comprising the steps of:
(1) moving the needle shield from the first shield position to the second shield position;
(2) actuating the actuation means such that a liquid medicament is delivered from the single-use injector; and
(3) upon completion of liquid medicament delivery, moving the needle shield from the second shield position to the third shield position.

25. A method of operating the needle safety shield assembly according to any one of the preceding claims comprising the steps of moving the needle shield to expose the needle for use; and moving the needle shield to re-shield the needle.

26. A method of operating a needle safety shield assembly for an injector, said needle safety shield comprising: a needle shield for shielding a needle of the injector; and
a shield locking means comprising a track adapted to engage with a runner, the track comprising at least one unidirectional barrier through which the runner can pass; said method comprising:
(a) moving the needle shield to expose the needle for use; and
(b) moving the needle shield to re-shield the needle,
wherein steps (a) and (b) cause movement of the runner along the track through the at least one unidirectional barrier and into a locked position in which further movement of the runner and needle shield is prevented.

27. A method of operating a needle safety shield assembly according to claim 4, comprising the steps of:
(a) moving the needle shield from the first shield position in which the needle is shielded to the second shield position in which the needle is exposed for use, wherein on movement between the first shield position and the second shield position the runner passes along the first track and through the first unidirectional barrier into the second track; and
(b) moving the needle shield from the second shield position to a third shield position in which the needle is re-shielded and the needle shield is prevented from further movement to expose the needle, wherein on movement between the second shield position and the third shield position the runner passes along the second track and through the second unidirectional barrier into a locked position in which further movement of the runner and needle shield is prevented.

28. A method of operating the needle safety shield assembly of claim 4, comprising the steps of:
(a) moving the needle shield from the first shield position in which the needle is shielded to the second shield position in which the needle is exposed for use, wherein on movement between the first shield position and the second shield position the runner passes along the first track and through the first unidirectional barrier into the second track; and
(b) moving the needle shield from the second shield position to a third shield position in which the needle is re-shielded and the needle shield is prevented from further movement to expose the needle; wherein on movement between the second shield position and the third shield position the runner passes along the second track and through the second unidirectional barrier into a locked position in which further movement of the runner and needle shield is prevented.
